(19) United States
(12) Patent Application Publication
(10) Pub. No.: US 2020/0377446 A1
(43) Pub. Date: Dec. 3, 2020

(54) METHOD FOR MANUFACTURING AROMATIC AZO COMPOUND

(71) Applicant: FUJIFILM Corporation, Tokyo (JP)

(72) Inventors: Masaya NAKAYAMA, Kanagawa (JP); Koji OMORI, Kanagawa (JP)

(73) Assignee: FUJIFILM Corporation, Tokyo (JP)

(21) Appl. No.: 16/882,737

(22) Filed: May 25, 2020

(30) Foreign Application Priority Data
May 30, 2019 (JP) 2019-101146

(51) Int. Cl. C07C 245/08

(52) U.S. Cl. CPC C07C 245/08

(57) ABSTRACT

Provided is a method for manufacturing an aromatic azo compound that is excellent in manufacturing efficiency and yield.

The method for manufacturing an aromatic azo compound includes obtaining an azoxy compound by heating a mixture containing nitrobenzenes, an aldose, and an alkaline substance to a temperature of 25° C. to 50° C. under an alkaline condition, and then manufacturing an aromatic azo compound by further heating the mixture, in which the azoxy compound is generated, to a temperature higher than 50° C.

### CROSS-REFERENCE TO RELATED APPLICATIONS

The present application claims priority under 35 U.S.C. § 119 to Japanese Patent Application No. 2019-101146, filed on May 30, 2019. The above application is hereby expressly incorporated by reference, in its entirety, into the present application.

### BACKGROUND OF THE INVENTION

### 1. Field of the Invention

The present invention relates to a method for manufacturing an aromatic azo compound.

### 2. Description of the Related Art

The azo compound has been used as a coloring agent or the like for a long time. In recent years, the azo compound has been used as the structure of a functional compound or a part thereof. For example, in J. Chem. Soc. Perkin Trans. II 1995, 1679, Macromolecules; vol. 43; nb. 3; (2010); p-1319-1328, and RSC Advances 2014, 4, 41371-41377, a case where the azo compound is used as a photoresponsive material is introduced.

### SUMMARY OF THE INVENTION

The inventors of the present invention studied the method for manufacturing an aromatic azo compound with reference to the above documents. As a result, the inventors have found that the manufacturing efficiency of the manufacturing methods described in the above documents is low overall (herein, “manufacturing efficiency” refers to the maximum amount of reactants per raw materials used) and needs to be improved. In addition, the inventors have found that the yield of the manufacturing methods described in the above documents also needs to be improved.

An object of the present invention is to provide a method for manufacturing an aromatic azo compound that is excellent in manufacturing efficiency and yield.

In order to achieve the above object, the inventors of the present invention carried out intensive examinations. As a result, the inventors have found that the object can be achieved by the following constitution.

[1] A method for manufacturing an aromatic azo compound, including obtaining an azoxy compound by heating a mixture containing nitrobenzenes, an aldose, and an alkaline substance to a temperature of 25° C. to 50° C. under an alkaline condition, and then manufacturing an aromatic azo compound by further heating the mixture, in which the azoxy compound is generated, to a temperature higher than 50° C.

[2] The method for manufacturing an aromatic azo compound described in [1], in which a mixing amount of the aldose with respect to the nitrobenzenes is 0.8 to 2.0 molar equivalents with respect to 1.0 molar equivalent of a nitro group, and a mixing amount of the alkaline substance with respect to the nitrobenzenes is 4.3 to 20.0 molar equivalents with respect to 1.0 molar equivalent of a nitro group.

[3] The method for manufacturing an aromatic azo compound described in [1] or [2], in which the nitrobenzenes are phthalic acids having a nitro group.

[4] The method for manufacturing an aromatic azo compound described in any one of [1] to [3], in which the nitrobenzenes are 5-nitroisophthalic acid.

[5] The method for manufacturing an aromatic azo compound described in any one of [1] to [4], in which in manufacturing the aromatic azo compound by heating the mixture, in which the azoxy compound is generated, to a temperature higher than 50° C., the aromatic azo compound is manufactured while air is being introduced into a reaction system.

[6] The method for manufacturing an aromatic azo compound described in [5], in which after the mixture, in which the azoxy compound is generated, is heated to a predetermined temperature higher than 50° C., air is introduced into the reaction system while the temperature is being maintained.

According to the present invention, it is possible to provide a method for manufacturing an aromatic azo compound which is excellent in manufacturing efficiency and yield.

### DESCRIPTION OF THE PREFERRED EMBODIMENTS

Hereinafter, the present invention will be specifically described.

The following constituents will be described based on typical embodiments of the present invention in some cases, but the present invention is not limited to the embodiments.

In the present specification, a range of numerical values described using “to” means a range including numerical values listed before and after “to” as a lower limit and an upper limit

[Method for Manufacturing Aromatic Azo Compound]

The method for manufacturing an aromatic azo compound according to an embodiment of the present invention (hereinafter, referred to as “the manufacturing method according to the embodiment of the present invention” as well) is a method for manufacturing an aromatic azo compound including obtaining an azoxy compound by heating a mixture containing nitrobenzenes, an aldose, and an alkaline substance to a temperature of 25° C. to 50° C. under an alkaline condition, and then manufacturing an aromatic azo compound by further heating the mixture, in which the azoxy compound is generated, to a temperature higher than 50° C.

Hereinafter, the manufacturing method according to the embodiment of the present invention will be described in comparison with the conventional manufacturing method. Hereinafter, an aspect, in which an azo dye A (corresponding to an aromatic azo compound) represented by the following structure is manufactured using 5-nitroisophthalic acid (hereinafter, referred to as “5-NIPA” as well) as nitrobenzenes and D-glucose as an aldose, will be described for example.

Furthermore, in the following description, the treatment for obtaining an azoxy compound by heating a mixture containing nitrobenzenes, an aldose, and an alkaline substances to a temperature of 25° C. to 50° C. under an alkaline condition will be referred to as step 1, and the treatment for manufacturing an aromatic azo compound by further heating the mixture obtained in the step 1 to a temperature higher than 50° C. will be referred to as step 2.

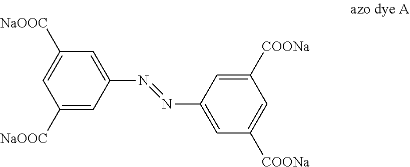

In a case where a mixture containing 5-NIPA, D-glucose, and an alkaline substance (for example, sodium hydroxide or the like) is heated to a temperature of 25° C. to 50° C. under an alkaline condition, due to the reduction reaction of the 5-NIPA by the D-glucose, an azoxy compound (intermediate A) represented by the following structural formula is generated. In other words, by performing the treatment as the step 1, the intermediate A represented by the following structural formula is generated. An aldehyde group in the D-glucose performs a reducing action on a nitro group of the 5-NIPA.

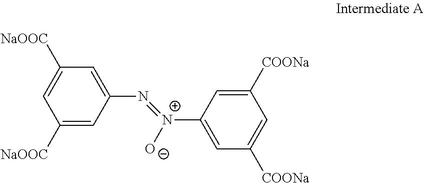

Then, the treatment (step 2) is performed in which the azo dye A is manufactured by further heating the mixture obtained in the step 1 to a temperature higher than 50° C. (that is, by further heating the mixture obtained in the step 1 to a temperature higher than 50° C. without isolating the intermediate A). In the step 2, a first reaction, in which an intermediate B (hydrazine compound) represented by the following structural formula is generated by reducing the intermediate A by using the D-glucose, and a second reaction, in which the aforementioned azo dye A is generated by oxidizing the intermediate B, proceed. The reduction reaction (first reaction) of the intermediate A by the D-glucose hardly proceeds under the temperature condition of the step 1 in which the temperature is equal to or lower than 50° C. That is, in the step 1, the generation of the intermediate B is inhibited, and the intermediate A is selectively generated.

In the first reaction, the aldehyde group in the D-glucose performs a reducing action on an azoxy moiety of the intermediate A.

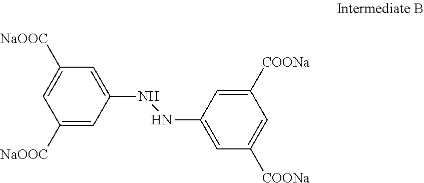

The first reaction is a reaction in which the intermediate B is generated by reducing the intermediate A by using the D-glucose. In the mixture obtained through the first reaction, the intermediate A and the intermediate B are mixed together. Furthermore, after the D-glucose in the mixture is almost exhausted with the generation of the intermediate B, the second reaction proceeds. The second reaction is a reaction in which the intermediate B is oxidized and converted into the azo dye A by the action of oxygen in the atmosphere, oxygen dissolved in the mixture, and the intermediate A in the mixture as an oxidant. In the second reaction, the intermediate A used as an oxidant in the reaction of conversion of the intermediate B into the azo dye A is also converted into the azo dye A just as the intermediate B described above.

Due to the constitution described above, the manufacturing method according to the embodiment of the present invention is excellent in manufacturing efficiency and yield.

The mechanism of action described above is unclear. It is considered that in the manufacturing method according to the embodiment of the present invention, at the stage following the first reaction, the ratio between the azoxy compound (corresponding to the intermediate A described above) and the hydrazine compound (corresponding to the intermediate B described above) generated in the reaction system is appropriately adjusted, and accordingly, in the second reaction, the azoxy compound may easily function as an oxidant in the reaction of oxidation of the hydrazine compound into the aromatic azo compound (corresponding to the azo dye A described above). Presumably, as a result, the conversion reaction to the aromatic azo compound in the step 2 may efficiently proceed, and the yield may be improved. Furthermore, the manufacturing method according to the embodiment of the present invention also has an advantage that the required mixing amount of the aldose is small. That is, as a result, the manufacturing efficiency (as described above, “manufacturing efficiency” means the maximum amount of reactants per raw materials used) also becomes excellent.

In contrast, in the conventional manufacturing method, an aromatic azo compound is manufactured by reacting nitrobenzenes, an aldose, and an alkaline substance at a temperature such as 70° C. without performing the treatment as the step 1. In the conventional manufacturing method, depending on the difference in the reaction rate, while an azoxy compound (corresponding to the intermediate A described above) is easily formed, it is difficult to control the reaction of generating a hydrazine compound (corresponding to the intermediate B described above) and an aromatic azo compound (corresponding to the azo dye A described above), and consequently, the yield and the purity are low. In a case where the mixing amount of the aldose is increased, the yield and the purity can be improved to some extent. However, because the required mixing amount of the aldose is increased, the manufacturing efficiency becomes poor.

In the manufacturing method according to the embodiment of the present invention, the mixing amount of the aldose with respect to nitrobenzenes is preferably 0.8 to 2.0 molar equivalents with respect to 1.0 molar equivalent of a nitro group, and the mixing amount of the alkaline substance with respect to the nitrobenzenes is preferably 4.3 to 20.0 molar equivalents with respect to 1.0 molar equivalent of a nitro group.

In a case where the mixing amount of the aldose with respect to nitrobenzenes is equal to or smaller than 2.0 molar equivalents with respect to 1.0 molar equivalent of a nitro group, the content of the azoxy compound remaining in the mixture at the stage following the first reaction becomes appropriate. Therefore, in the second reaction, the azoxy compound easily functions as an oxidant for the reaction of oxidation of the hydrazine compound to the aromatic azo compound. In contrast, in a case where the mixing amount of the aldose is equal to or greater than 0.8 molar equivalents with respect to 1.0 molar equivalent of the nitrobenzenes, each reaction in the step 1 and the step 2 more appropriately proceeds.

In a case where the mixing amount of the alkaline substance with respect to the nitrobenzenes is equal to or greater than 4.3 molar equivalents with respect to 1.0 molar equivalent of a nitro group, the reaction rate in each of the treatments as the step 1 and the step 2 increases, the ratio between the azoxy compound and the hydrazine compound generated after the first reaction of the step 2 can be appropriately adjusted, and as a result, the purity and the yield can be further improved. The upper limit of the mixing amount of the alkaline substance with respect to the nitrobenzenes is not particularly limited, but is, for example, equal to or smaller than 20.0 molar equivalents with respect to 1.0 molar equivalent of a nitro group.

That is, in the manufacturing method according to the embodiment of the present invention, from the viewpoint of temperature control at the time of performing the treatments as the step 1 and the step 2 described above and from the viewpoint of the mixing amount of each of the nitrobenzenes, the aldose, and the alkaline substance, the mixing amount is adjusted such that the azoxy compound easily functions as an oxidant for the reaction of oxidation of the hydrazine compound into the aromatic azo compound during the second reaction of the step 2.

Hereinafter, the manufacturing method according to the embodiment of the present invention will be specifically described.

[Nitrobenzenes]

Nitrobenzenes mean compounds obtained in a case where carbon atoms as members of a benzene ring are substituted with one or more nitro groups. Examples of the nitrobenzenes include a compound represented by the following General Formula (1).

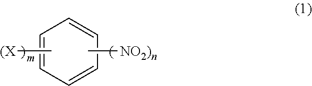

In General Formula (1), X represents a substituent. m represents an integer of 0 to 5. n represents 1 or 2. m+n is equal to or smaller than 6. In a case where m is an integer equal to or greater than 2, a plurality of Xs may be the same as or different from each other.

The substituent represented by X is not particularly limited, and examples thereof include an alkyl group, an alkoxy group, an aryl group, a heteroaryl group, a carboxy group, a hydroxy group, a halogen atom, and an amino group (—NH₂).

The number of carbon atoms in the alkyl group and the alkoxy group is, for example, preferably 1 to 6.

The number of carbon atoms contained in the aryl group is preferably 6 to 10, and more preferably 6. The aromatic hydrocarbon ring constituting the aryl group may have a monocyclic structure or a condensed ring structure. The aryl group may further have a substituent.

The aromatic heterocyclic ring constituting the heteroaryl group is preferably a 5- to 7-membered ring having at least one nitrogen atom, oxygen atom, sulfur atom, or selenium atom in the ring structure, and more preferably a 5- or 6-membered ring having at least one nitrogen atom, oxygen atom, sulfur atom, or selenium atom in the ring structure. The aromatic heterocyclic ring may be a monocyclic ring or a condensed ring structure. In addition, the heteroaryl group may further have a substituent.

Examples of the halogen atom include a fluorine atom, a chlorine atom, a bromine atom, and an iodine atom.

In a case where m is an integer equal to or greater than 2, a plurality of Xs may be the same as or different from each other.

Particularly, X is preferably a carboxy group or a hydroxy group, and more preferably a carboxy group.

m is preferably 1 to 3, more preferably 1 or 2, and even more preferably 2.

n is preferably 1.

The nitrobenzenes are not particularly limited, and examples thereof include nitrobenzene, nitrotoluenes such as 2-nitrotoluene, 3-nitrotoluene, and 4-nitrotoluene; ethyl nitrobenzenes such as 2-ethylnitrobenzene, 3-ethylnitrobenzene, and 4-ethylnitrobenzene; propylnitrobenzenes such as 2-propylnitrobenzene, 3-propylnitrobenzene, and 4-propylnitrobenzene; nitrocumene such as 2-nitrocumene, 3-nitrocumene, and 4-nitrocumene; t-butylnitrobenzenes such as 1-butylnitrobenzene, 1-t-butyl-3-nitrobenzene, 1-t-butyl-4-nitrobenzene, and tri-t-butylnitrobenzene; fluoronitrobenzenes such as 2-fluoronitrobenzene, 3-fluoronitrobenzene, and 4-fluoronitrobenzene; chloronitrobenzenes such as 2-chloronitrobenzene, 3-chloronitrobenzene, and 4-chloronitrobenzene; bromobenzenes such as 2-bromonitrobenzene, 3-bromonitrobenzene, and 4-nitronitrobenzene; iodonitrobenzenes such as 2-iodonitrobenzene, 3-iodonitrobenzene, and 4-iodonitrobenzene; nitrobenzoic acids such as 2-nitrobenzoic acid, 3-nitrobenzoic acid, and 4-nitrobenzoic acid; 2-nitroisophthalic acid; 5-nitroisophthalic acid (5-NIPA); monomethyl 5-nitroisophthalate; nitroterephthalic acid; nitrobiphenyl carboxylic acid; dinitrohalobenzenes such as dinitrofluorobenzene, dinitrochlorobenzene, dinitrobromobenzene, and dinitroiodobenzene; 3,5-dinitrobenzoic acid; 2,4-dinitrobenzoic acid; 4-methyl-3,5-dinitrobenzoic acid; 2-hydroxy-3,5-dinitro benzoic acid; and the like.

Particularly, as the nitrobenzenes, phthalic acids having a nitro group are preferable, and 5-nitroisophthalic acid (5-NIPA) is more preferable.

[Aldose]

The aldose means a monosaccharide which is represented by CₙH₂ₙOₙ (n is an integer equal to or greater than 3) and has one aldehyde group on a terminal. The aldehyde group in the aldose performs a reducing action on the nitro group of the nitrobenzenes and on the azoxy moiety of the azoxy compound obtained by the step 1.

Examples of the aldose include glyceraldehyde, erythrose, threose, ribose, arabinose, xylose, lyxose, allose, altrose, glucose, mannose, gulose, idose, galactose, talose, and the like. Among these, glucose is preferable.

The mixing amount of the aldose with respect to the nitrobenzenes is preferably 0.8 to 2.0 molar equivalents, more preferably 1.0 to 1.8 molar equivalents, and even more preferably 1.0 to 1.5 molar equivalents with respect to 1.0 molar equivalent of the nitro group.

[Alkaline Substance]

The alkaline substance is not particularly limited as long as the pH thereof becomes higher than 7 in a case where the alkaline substance is dissolved in water. Specifically, examples of the alkaline substance include sodium hydroxide, potassium hydroxide, lithium hydroxide, rubidium hydroxide, cesium hydroxide, magnesium hydroxide, calcium hydroxide, sodium carbonate, potassium carbonate, lithium carbonate, rubidium carbonate, cesium carbonate, magnesium carbonate, calcium carbonate, sodium hydrogen carbonate, potassium hydrogen carbonate, lithium hydrogen carbonate, rubidium hydrogen carbonate, and cesium hydrogen carbonate. Among these, sodium hydroxide, potassium hydroxide, sodium carbonate, potassium carbonate, sodium hydrogen carbonate, or potassium hydrogen carbonate is preferable, and sodium hydroxide or potassium hydroxide is more preferable.

The mixing amount of the alkaline substance with respect to the nitrobenzenes is 4.3 to 20.0 molar equivalents, preferably 4.4 to 8.0 molar equivalents, and more preferably 4.5 to 5.0 molar equivalents with respect to 1.0 molar equivalent of the nitro group.

[Aromatic Azo Compound]

The aromatic azo compound obtained by the manufacturing method according to the embodiment of the present invention is not particularly limited, but is preferably, for example, a compound represented by the following General Formula (2).

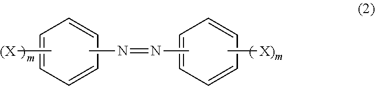

In General Formula (2), X represents a substituent. m represents an integer of 0 to 5. In a case where there is a plurality of Xs, the plurality of Xs may be the same as or different from each other.

In General Formula (2), X and m have the same definitions as X and m in General Formula (1), and the suitable embodiments thereof are also the same.

[Manufacturing Procedure]

The manufacturing method according to the embodiment of the present invention includes a step 1 of obtaining an azoxy compound by heating a mixture containing nitrobenzenes, an aldose, and an alkaline substance to a temperature of 25° C. to 50° C. under an alkaline condition, and a step 2 of manufacturing an aromatic azo compound by further heating the mixture containing the azoxy compound obtained through the step 1 to a temperature higher than 50° C.

Furthermore, as will be described later, in the manufacturing method according to the embodiment of the present invention, in a case where the aromatic azo compound is manufactured by heating the mixture to a temperature higher than 50° C. in the step 2, it is preferable to manufacture the aromatic azo compound while air is being introduced into the reaction system. In a case where air is allowed to present in the reaction system as an oxidant during the reaction (second reaction) in the step 2 in which the aromatic azo compound is generated, the reaction can be further accelerated, and consequently, the aromatic azo compound can be manufactured with higher yield and higher purity.

<Step 1>

In a case where the step 1 is performed as described above as an example of the aspect of manufacturing the azo dye A by using 5-nitroisophthalic acid (5-NIPA) as nitrobenzenes and D-glucose as an aldose, through the reduction reaction of the nitrobenzenes by the aldose, an azoxy compound is generated.

Specifically, the step 1 is preferably a step of stirring a mixture, which is obtained by dissolving and/or dispersing components such as nitrobenzenes, an aldose, and an alkaline substance in a solvent, at a temperature of 25° C. to 50° C. under an alkaline condition.

The solvent is not particularly limited, and examples thereof include water; glycols such as ethylene glycol and propylene glycol; cyclic ethers such as tetrahydrofuran and tetrahydropyran; formamides such as dimethylacetamide and dimethylformamide; ketones such as acetone and 2-butanone; nitriles such as acetonitrile; and the like. Among these, water is preferable.

These solvents may be used singly or used by being mixed together.

In the above mixture, the amount of the solvent used with respect to the mass of the nitrobenzenes is preferably equal to or greater than 3.0% by mass, and more preferably equal to or greater than 20.0% by mass. The upper limit thereof is preferably equal to or smaller than 80.0% by mass, and more preferably equal to or smaller than 40.0% by mass.

The step 1 is performed under an alkaline condition. That is, in the suitable embodiments described above, the pH of the mixture is alkaline.

The pH of the alkaline condition in the step 1 is not particularly limited as long as it is higher than 7.0. However, the pH of the alkaline condition is preferably equal to or higher than 11.0, because then the reaction rate of the step 1 is further improved, and hence the yield is further increased. The upper limit thereof is not particularly limited, but is equal to or lower than 14.0 in many cases.

In view of further improving the yield and the purity, the reaction temperature in the step 1 is preferably 30° C. to 50° C.

Furthermore, the stirring time in the step 1 is preferably, for example, 0.5 to 5 hours.

<Step 2>

The step 2 is a step of performing a treatment for manufacturing an aromatic azo compound by further heating the mixture obtained in the step 1 to a temperature higher than 50° C. under an alkaline condition.

In a case where the step 2 is performed as described above as an example of the aspect of manufacturing the azo dye A by using 5-nitroisophthalic acid (5-NIPA) as nitrobenzenes and D-glucose as an aldose, a first reaction, in which the azoxy compound obtained by the step 1 is reduced by the aldose and a hydrazine compound is generated, and a second reaction, in which the hydrazine compound obtained by the first reaction is oxidized and an aromatic azo compound is generated, occur.

The step 2 is performed under an alkaline condition. That is, in the suitable embodiments described above, the pH of the mixture is alkaline.

The pH of the alkaline condition in the step 2 is not particularly limited as long as it is higher than 7.0. However, the pH of the alkaline condition is preferably equal to or higher than 11.0, because then the reaction rate of the step 2 is further improved, and hence the yield is further increased. The upper limit thereof is not particularly limited, but is equal to or lower than 14.0 in many cases.

In view of further improving the yield and the purity, the reaction temperature in the step 2 is preferably equal to or higher than 60° C. The upper limit of the reaction temperature is not particularly limited. However, in view of preventing the reaction from proceeding too rapidly, the upper limit thereof is preferably equal to or lower than 100° C.

In the step 2, after the mixture reaches a predetermined temperature, it is preferable to further stir the mixture for 1 to 72 hours, for example.

Furthermore, in a case where the reaction slowly proceeds in the step 2, in order to accelerate the reaction, an oxidant may be added. As the oxidant, oxygen, hydrogen peroxide, percarboxylic acids such as metachloroperbenzoic acid and peracetic acid, N-methylmorpholine-N-oxide, manganese dioxide, a chromium-based oxidant such as Jones reagent or Pyridinium Dichromate (PDC), an iodine-based oxidant such as Dess-Martin reagent, 2,2,6,6-tetramethylpiperidine-1-oxyl (TEMPO), or dimethyl sulfoxide is preferable, and oxygen or air is more preferable.

Among these, from the viewpoint of economics, air is even more preferable as the oxidant. In a case where the aromatic azo compound is manufactured by heating the mixture to a temperature higher than 50° C. in the step 2, it is preferable to manufacture the aromatic azo compound while air is being introduced into the reaction system. Regarding the procedure of introducing air, air may be introduced into the reaction system while the mixture is being heated to a predetermined temperature higher than 50° C., or, the mixture may be heated to a predetermined temperature higher than 50° C. and then air may be introduced into the reaction system while the temperature is being maintained. Particularly, it is preferable to heat the mixture to a predetermined temperature higher than 50° C. and then introduce air into the reaction system while the temperature is being maintained, because then the aromatic azo compound can be manufactured with a higher yield and higher purity.

In a case where a gas such as oxygen or air is introduced into the reaction system as an oxidant, the introduction method is not particularly limited, and examples thereof include a method using a gas injection nozzle.

[Use]

The aromatic azo compound obtained by the manufacturing method according to the embodiment of the present invention can be used, for example, as a ligand of Metal-Organic Frameworks (MOF).

### EXAMPLES

Hereinafter, the present invention will be more specifically described based on examples. The materials, the amount and ratio of the materials used, how to treat the materials, the treatment procedure, and the like shown in the following examples can be appropriately changed as long as the gist of the present invention is maintained. Therefore, the scope of the present invention is not limited to the following examples.

Various components used in Examples are as follows.

5-Nitroisophthalic acid (corresponding to “5-NIPA” in Table 1): manufactured by TOKYO CHEMICAL INDUSTRY CO., LTD.

4-Nitrobenzoic acid: manufactured by FUJIFILM Wako Pure Chemical Corporation

Sodium hydroxide (NaOH): manufactured by FUJIFILM Wako Pure Chemical Corporation

Potassium hydroxide (KOH): manufactured by FUJIFILM Wako Pure Chemical Corporation

D-glucose (corresponding to “glucose” in Table 1): manufactured by FUJIFILM Wako Pure Chemical Corporation

Water: distilled water

[1] Manufacturing of Aromatic Azo Compound

### Example 1

5-Nitroisophthalic acid (30 g (0.14 mol)), 27.0 g (0.68 mol) of NaOH, and 25.5 g (0.14 mol) of D-glucose are added to 151.5 g of water and stirred. The mixture is stirred at a temperature of 40° C. to 50° C. (see “Temperature condition 1” in Table 1) for 1 hour (corresponding to “step 1”) and then heated to 70° C. (see “Temperature condition 2” in Table 1). After reaching the temperature, the mixture is stirred at 70° C. (“Temperature condition 2”) for 4 hours (corresponding to “step 2”). Thereafter, the mixture is cooled to room temperature and filtered, thereby obtaining a target substance. The obtained Wet crystals are dried in a dryer at 50° C. and taken out at a point in time when the temperature becomes constant.

### Example 2

An aromatic azo compound is manufactured by the same method as the manufacturing method of Example 1, except that the mixing amount of the D-glucose with respect to the 5-nitroisophthalic acid and the mixing amount of the NaOH with respect to the 5-nitroisophthalic acid are changed to the numerical values shown in Table 1.

### Example 3

An aromatic azo compound is manufactured by the same method as the manufacturing method of Example 1, except that the 5-nitroisophthalic acid is replaced with 4-nitrobenzoic acid.

### Example 4

An aromatic azo compound is manufactured by the same method as the manufacturing method of Example 1, except that NaOH is replaced with KOH.

### Example 5

An aromatic azo compound is manufactured by the same method as the manufacturing method of Example 1, except that the mixing amount of the D-glucose with respect to the 5-nitroisophthalic acid, the mixing amount of NaOH with respect to the 5-nitroisophthalic acid, and the mixing amount of water are changed to the numerical values shown in Table 1.

### Example 6

5-Nitroisophthalic acid (30 g (0.14 mol)), 27.0 g (0.68 mol) of NaOH, and 33.3 g (0.18 mol) of D-glucose are added to 160 g of water and stirred. The mixture is stirred at a temperature of 40° C. to 50° C. (see “Temperature condition 1” in Table 1) for about 1 hour (corresponding to “step 1”) and then heated to 70° C. (see “Temperature condition 2” in Table 1). After reaching the temperature, the mixture is kept at 70° C. (“Temperature condition 2”), and in this state, air is blown into the reaction vessel and stirred for 4 hours (corresponding to “step 2”). Thereafter, the mixture is cooled to room temperature and filtered, thereby obtaining a target substance. The obtained Wet crystals are dried in a dryer at 50° C. and taken out at a point in time when the temperature becomes constant.

### Comparative Examples 1 to 4

5-Nitroisophthalic acid, NaOH, D-glucose, and water in the mixing amounts shown in Table 1 are mixed together at 70° C., and the mixture is stirred for several hours while being kept at 70° C. After the stirring is finished, the mixture is cooled to room temperature and filtered, thereby obtaining a target substance. The obtained Wet crystals are dried in a dryer at 50° C. and taken out at a point in time when the temperature becomes constant.

### Comparative Example 5

By using 5-nitroisophthalic acid, NaOH, D-glucose, and water, an aromatic azo compound is manufactured by the manufacturing method of Macromolecules; vol. 43; nb. 3; (2010); p-1319-1328.

### Comparative Example 6

By using 4-nitrobenzoic acid, NaOH, D-glucose, and water, an aromatic azo compound is manufactured by the manufacturing method of J. Chem. Soc. Perkin Trans. II 1995, 1679.

### Comparative Example 7

By using 5-nitroisophthalic acid, NaOH, D-glucose, and water, an aromatic azo compound is manufactured by the manufacturing method of RSC Advances 2014, 4, 41371-41377.

[Evaluation Results]

Table 1 collectively shows the yield (%), the purity (%), and the manufacturing efficiency of the manufacturing methods of each of the examples and each of the comparative examples. “Manufacturing efficiency” means a value obtained by dividing “total liquid amount of mixture” by “amount of nitrobenzenes used”.

From the viewpoint of practical use, the yield (%) is preferably equal to or higher than 60%, and more preferably equal to or higher than 70%. From the viewpoint of practical use, the purity (%) is preferably equal to or higher than 85%, and more preferably equal to or higher than 90%. From the viewpoint of practical use, the manufacturing efficiency is preferably equal to or lower than 8.

In Table 1, “A” in “Temperature condition” in the column of “Step 1” means that the temperature in the step 1 is in a range of 25° C. to 50° C., and “B” in the same column means that the temperature in the step 1 is in a range of lower than 20° C. and higher than 50° C. Furthermore, “A” in “Temperature condition” in the column of “step 2” means that the temperature in the step 2 is in a range higher than 50° C.

In Table 1, “mixing amount of water (w/w)” means a numerical value determined by (amount of used water)÷(amount of used nitrobenzenes).

TABLE 1

| | Nitrobenzenes | | | | | | | | | | | | | |
|---|---|---|---|---|---|---|---|---|---|---|---|---|---|---|
| | | Molar | | | Alkaline | | | Step 1 | | Step 2 | | Evaluation result | | |
| | | equiv- | Aldose | | substance | | Water | Temper- | | Temper- | | | | Manufac- |
| | | alent | | Molar | | Molar | Mixing | ature | | ature | | | | turing |
| | | of nitro | | equiv- | | equiv- | amount | condi- | | condi- | | Yield | Purity | effi- |
| | Type | group | Type | alent | Type | alent | (w/w) | tion | pH | tion | pH | (%) | (%) | ciency |
| Example 1 | 5-NIPA | 1.0 | Glucose | 1.0 | NaOH | 4.9 | 5.1 | A | >7.0 | A | >7.0 | 100 | 95 | 8 |
| Example 2 | 5-NIPA | 1.0 | Glucose | 1.1 | NaOH | 4.8 | 5.1 | A | >7.0 | A | >7.0 | 81 | 96 | 8 |
| Example 3 | 4-Nitro- | 1.0 | Glucose | 1.0 | NaOH | 4.9 | 5.1 | A | >7.0 | A | >7.0 | 84 | 96 | 8 |
| | benzoic | | | | | | | | | | | | | |
| | acid | | | | | | | | | | | | | |
| Example 4 | 5-NIPA | 1.0 | Glucose | 1.0 | KOH | 4.9 | 5.1 | A | >7.0 | A | >7.0 | 81 | 93 | 8 |
| Example 5 | 5-NIPA | 1.0 | Glucose | 0.9 | NaOH | 5.3 | 5.6 | A | >7.0 | A | >7.0 | 70 | 90 | 8 |
| Example 6 | 5-NIPA | 1.0 | Glucose | 1.3 | NaOH | 4.9 | 5.3 | A | >7.0 | A | >7.0 | 95 | 97 | 8 |
| Comparative | 5-NIPA | 1.0 | Glucose | 1.7 | NaOH | 14.0 | 12.5 | B | >7.0 | A | >7.0 | 100 | 95 | 15 |
| Example 1 | | | | | | | | | | | | | | |
| Comparative | 5-NIPA | 1.0 | Glucose | 1.8 | NaOH | 20.0 | 14.5 | B | >7.0 | A | >7.0 | 67 | 99 | 15 |
| Example 2 | | | | | | | | | | | | | | |
| Comparative | 5-NIPA | 1.0 | Glucose | 1.0 | NaOH | 4.9 | 5.1 | B | >7.0 | A | >7.0 | 61 | 87 | 8 |
| Example 3 | | | | | | | | | | | | | | |
| Comparative | 5-NIPA | 1.0 | Glucose | 1.4 | NaOH | 14.0 | 15.5 | B | >7.0 | A | >7.0 | 100 | 94 | 15 |
| Example 4 | | | | | | | | | | | | | | |
| Comparative | 5-NIPA | 1.0 | Glucose | 2.1 | NaOH | 6.0 | 26 | B | >7.0 | A | >7.0 | 61 | 95 | 30 |
| Example 5 | | | | | | | | | | | | | | |
| Comparative | 4-Nitro- | 1.0 | Glucose | 6.2 | NaOH | 13.9 | 25 | B | >7.0 | A | >7.0 | 72 | 95 | 36 |
| Example 6 | benzoic | | | | | | | | | | | | | |
| | acid | | | | | | | | | | | | | |
| Comparative | 5-NIPA | 1.0 | Glucose | 6.6 | NaOH | 8.1 | 17 | B | >7.0 | A | >7.0 | 70 | 95 | 25 |
| Example 7 | | | | | | | | | | | | | | |

From the results shown in Table 1, it has been revealed that according to the manufacturing method of the embodiment of the present invention, it is possible to manufacture an aromatic azo compound with a higher yield and better manufacturing efficiency.

Furthermore, from the results of Examples 1 to 5, it has been revealed that in a case where the mixing amount of the aldose with respect to the nitrobenzenes is equal to or greater than 1.0 molar equivalent with respect to 1.0 molar equivalent of a nitro group, it is possible to manufacture an aromatic azo compound with a higher yield and higher purity. Presumably, because the reaction rate of the reaction (first reaction) in the step 2, in which the hydrazine compound is generated, may become more appropriate, the above result may be obtained.

In addition, by the comparison of Examples 1 to 6, it has been revealed that in a case where air is introduced as an oxidant during the reaction (second reaction) in the step 2, in which an aromatic azo compound is generated, so as to further accelerate the reaction, it is possible to manufacture an aromatic azo compound with a higher yield and higher purity.

On the other hand, it has been revealed that by the manufacturing methods of the comparative examples, a high yield and an excellent manufacturing efficiency cannot be simultaneously achieved.

What is claimed is:

1. A method for manufacturing an aromatic azo compound, comprising:
obtaining an azoxy compound by heating a mixture containing nitrobenzenes, an aldose, and an alkaline substance to a temperature of 25° C. to 50° C. under an alkaline condition; and
then manufacturing an aromatic azo compound by further heating the mixture, in which the azoxy compound is generated, to a temperature higher than 50° C.

2. The method for manufacturing an aromatic azo compound according to claim 1,
wherein a mixing amount of the aldose with respect to the nitrobenzenes is 0.8 to 2.0 molar equivalents with respect to 1.0 molar equivalent of a nitro group, and
a mixing amount of the alkaline substance with respect to the nitrobenzenes is 4.3 to 20.0 molar equivalents with respect to 1.0 molar equivalent of a nitro group.

3. The method for manufacturing an aromatic azo compound according to claim 1,
wherein the nitrobenzenes are phthalic acids having a nitro group.

4. The method for manufacturing an aromatic azo compound according to claim 1,
wherein the nitrobenzenes are 5-nitroisophthalic acid.

5. The method for manufacturing an aromatic azo compound according to claim 1,
wherein in manufacturing the aromatic azo compound by heating the mixture, in which the azoxy compound is generated, to a temperature higher than 50° C., the aromatic azo compound is manufactured while air is being introduced into a reaction system.

6. The method for manufacturing an aromatic azo compound according to claim 5,
wherein after the mixture, in which the azoxy compound is generated, is heated to a predetermined temperature higher than 50° C., air is introduced into the reaction system while the temperature is being maintained.

7. The method for manufacturing an aromatic azo compound according to claim 2,
wherein the nitrobenzenes are phthalic acids having a nitro group.

8. The method for manufacturing an aromatic azo compound according to claim 2,
wherein the nitrobenzenes are 5-nitroisophthalic acid.

9. The method for manufacturing an aromatic azo compound according to claim 3,
wherein the nitrobenzenes are 5-nitroisophthalic acid.

10. The method for manufacturing an aromatic azo compound according to claim 2,
wherein in manufacturing the aromatic azo compound by heating the mixture, in which the azoxy compound is generated, to a temperature higher than 50° C., the aromatic azo compound is manufactured while air is being introduced into a reaction system.

11. The method for manufacturing an aromatic azo compound according to claim 3,
wherein in manufacturing the aromatic azo compound by heating the mixture, in which the azoxy compound is generated, to a temperature higher than 50° C., the aromatic azo compound is manufactured while air is being introduced into a reaction system.

12. The method for manufacturing an aromatic azo compound according to claim 4,
wherein in manufacturing the aromatic azo compound by heating the mixture, in which the azoxy compound is generated, to a temperature higher than 50° C., the aromatic azo compound is manufactured while air is being introduced into a reaction system.
